(19) United States
(12) Patent Application Publication
(10) Pub. No.: US 2011/0218635 A1
(43) Pub. Date: Sep. 8, 2011

(54) MODULAR KNEE IMPLANTS

(76) Inventors: Andrew Arthur Amis, London (GB); Robert Michael Wozencroft, Surrey (GB); Justin Peter Cobb, London (GB)

(21) Appl. No.: 13/003,384

(22) PCT Filed: Jul. 10, 2009
(86) PCT No.: PCT/GB2009/050823

§ 371 Date: Apr. 8, 2011

(30) Foreign Application Priority Data
Jul. 10, 2008 (GB) 0812631.0

(51) Int. Cl. A61F 2/38; A61F 2/30

(52) U.S. Cl. 623/20.18; 623/18.11; 623/20.32; 623/20.21; 623/20.28

(57) ABSTRACT

An implant for bone re-surfacing in a joint, the implant having a bearing platform having a front surface which forms a bearing surface and a back surface, and securing apparatus projecting from the back surface, the securing apparatus having a locking surface arranged to bear against an undercut surface of the bone to secure the implant against the bone.

### FIELD OF THE INVENTION

The present invention relates to knee implants and in particular to modular knee implants.

### BACKGROUND TO THE INVENTION

There is an increasing demand for surgical procedures to remedy pain caused by early stage arthritis in the knee, but due to the problems of implant wear and osteolysis, implants are not always expected to last a life time. Younger, highly active individuals who just want to maintain their lifestyle and overweight people, who wear out their joints quicker, pose a particular challenge to the modern orthopaedic surgeon.

Ideally the treatment of an individual should be managed carefully throughout an often long and active life by using more conservative implant devices and conserving natural tissue and bone where possible. This has two benefits; firstly it enables more natural movement and a return to normal activities and secondly it improves the chances of a successful re-operation at later stage.

The choice of bone conserving and soft tissue conserving implants are limited and because they must work in harmony with natural tissue, the surgical techniques are technically challenging and difficult to master. The devices that do exist such as uni-condylar and patello-femoral knee replacements have historically only achieved modest success, mainly due to technical difficulties. They often have limited indications and are not designed to be compatible with one another. Due to these drawbacks, most surgeons favour Total Knee Replacement (TKR) for all their patients because it is easier to achieve consistent results. However it is at the expense of removing excessive amounts of bone and sometimes perfectly healthy knee ligaments, severely limiting future surgical options.

Nevertheless, there is renewed interest in partial knee replacements, firstly because the implant components are smaller, they can be inserted through smaller incisions, and they therefore lend themselves to Minimally Invasive Surgery (MIS). MIS causes fewer traumas to the surrounding muscles and allows a more speedy recovery and discharge from hospital. However the technical difficulties are even greater than conventional surgery because the surgeon's access and visibility are impaired. Secondly, accuracy and reproducibility have been somewhat improved in recent years with the use of computer assisted navigation in surgery. This enables more accurate placement of the implant components in relation to joint surfaces and ligaments, even where MIS is employed. Navigation often uses pre-operative scans to accurately simulate the joint anatomy during surgery.

All existing knee replacement implants are inserted using sets of surgical instruments and surgical power tools to shape and prepare the bone surfaces. Even where navigation is employed, most of these devices are still needed. The most commonly used tool is the powered oscillating saw, which is used to remove entire segments of bone from the joint surfaces. It is only capable of making flat cuts, so it is no coincidence that knee implant components have predominantly flat underside surfaces to mate with these flat bone cuts. Furthermore because the joint surfaces are curved, but cuts are flat, implant components are often thicker than is necessary for strength, in order to make them flat on one side, as shown in FIGS. 1a and 1b, and can make the tibia liable to fracture as shown in FIG. 1c. The optimum shape for adequate strength and to conserve bone would be a constant cross section, with the inner surface curved and offset from the outer surface, as shown in FIGS. 1d and 1e, but this would not be compatible with the oscillating saw technique. Consequently the surgical technique and particularly the oscillating saw has influenced the design of modern partial and total knee replacement devices, causing compromise both in terms of the excessive amount of bone removed and the bulkiness of the implants. Where curved inner surfaces do exist, such as on some patello-femoral devices, free-hand nibbling or burring technique are used to shape the bone, which are inconsistent and not conducive with achieving the required accuracy.

A further technological advance in recent years is the employment of robotic techniques to further improve joint replacement surgery. Still in their infancy, these systems combine navigated pre-operative scanning based technology with a robot to assist the surgeon in preparing the joint surfaces during surgery. An example of such a system is the Acrobot Sculptor (The Acrobot Company Ltd, London UK). It employs a high speed burr attachment to ‘sculpt’ the bone surfaces. The computer controls the extent of the bone shaping within ‘Active Constraints’ so that it is not possible to cut outside a pre-defined volume. This allows very accurate shaping of the bone surfaces to mate with the implant components. There is no need for an oscillating saw or any of the instruments associated with a conventional technique.

This technique offers more flexibility in terms of the shapes that can be sculpted into the bone surfaces, but it has only been used with existing implants, designed for conventional surgical instruments and tools, so this new flexibility has not been explored.

### SUMMARY OF THE INVENTION

In view of the new bone shaping methods available, new possibilities for knee implant design which can be provided by the present invention are wide. For example, distinct pockets can be created in the bone surfaces to accept smaller partial implant components, targeting only those areas affected by cartilage erosion and wear. Recessing an implant component into a pocket surrounded by a natural bone edges can also enhance fixation by preventing sideways movement and rotation. Furthermore the specific requirements of an individual joint can be addressed by selecting a certain combination of components or even manufacturing a patient specific ‘set’. Whether patient specific or not, the implants can be minimal in size and optimised for bone conserving and soft tissue conserving techniques.

An aim of some embodiments of the present invention is to consider the optimum design for a suite of knee joint resurfacing implant components for robot assisted surgical techniques.

According to one aspect of the present invention there is provided an implant for bone re-surfacing in a joint, the implant comprising a bearing portion having a front surface which forms a bearing surface and a back surface, and securing means projecting from the back surface, the securing means having a locking surface arranged to bear against an undercut surface of the bone to secure the implant against the bone.

According to a further aspect of the invention there is provided a method of resurfacing a bone comprising cutting an undercut groove in the bone, providing an implant comprising a bearing portion with a back surface and securing means projecting from the back surface, the securing means having a locking surface arranged to bear against an undercut surface of the groove, and inserting the securing means into the groove to secure the implant against the bone.

The method may further comprise cutting a pocket into the bone into which the implant can be placed, the pocket having at least one side against which the implant can abut when fully inserted. For example where the implant is inserted into a tibial plateau in the anterior-posterior direction, the side of the pocket may be at the posterior end of the pocket.

Preferred embodiments of the present invention will now be described by way of example only with reference to the remainder of the accompanying drawings.

### BRIEF DESCRIPTION OF THE DRAWINGS

FIG. 1a is a section through a known knee implant set;

FIG. 1b is a front view of the knee implant set of FIG. 1a;

FIG. 1c is a front view of the tibial components of the implant set of FIG. 1a showing possible fracture of the tibia;

FIG. 1d is a schematic section through an idealised knee implant set;

FIG. 1e is a front view of the knee implant set of FIG. 1d;

FIG. 2 is a front view of a knee implant set according to a first embodiment of the invention;

FIG. 3 is a top view of the implant set of FIG. 2;

FIG. 4 is a view from the front and below of the implant set of FIG. 2;

FIG. 5 is a perspective view of the medial parts of the implant set of FIG. 2;

FIG. 6 is a top view of the medial parts of the implant set of FIG. 2;

FIG. 7 is a view from the medial side of the medial parts of the implant set of FIG. 2;

FIG. 8 is a posterior view of the medial parts of the implant set of FIG. 2;

FIG. 9 is a section on line A-A of FIG. 6;

FIG. 10 is a section on line C-C of FIG. 6;

FIG. 11 is a perspective view of the lateral parts of the implant set of FIG. 2;

FIG. 12 is a top view of the lateral parts of the implant set of FIG. 2;

FIG. 13 is a view from the lateral side of the lateral parts of the implant set of FIG. 2;

FIG. 14 is a posterior view of the lateral parts of the implant set of FIG. 2;

FIG. 15 is a view in the direction of arrow D of FIG. 13;

FIG. 16 is a section on line A-A of FIG. 12;

FIG. 17 is a section on line B-B of FIG. 13;

FIG. 18 is a perspective view of the patello-femoral parts of the implant set of FIG. 2;

FIG. 19 is a top view of the patello-femoral parts of the implant set of FIG. 2;

FIG. 20 is an anterior view of the patello-femoral parts of the implant set of FIG. 2;

FIG. 21 is a lateral side view of the patello-femoral parts of the implant set of FIG. 2;

FIG. 22 is a section on line A-A of FIG. 20;

FIG. 23 is a section on line B-B of FIG. 20;

FIG. 24 is a perspective view of the implant set of FIG. 2 when implanted into a knee;

FIG. 25 is a front view of the implant set of FIG. 2 when implanted into a knee;

FIG. 26 is a plan view of the tibial implants of the set of FIG. 2 when implanted into a tibia;

FIG. 26a is a plan view similar to FIG. 26 showing the articulation movement of the femur on the tibia;

FIG. 27 is a side view showing insertion of the medial tibial implant into the tibia;

FIG. 28 is a side view showing insertion of the lateral tibial implant into the tibia;

FIG. 29 is a schematic view of a bone shaping system for use in conjunction with the implants of FIGS. 1 to 28;

FIG. 30 is a section through a tibial implant according to a further embodiment of the invention;

FIG. 31 is a section through a tibial implant according to a further embodiment of the invention;

FIG. 32 is a front perspective view of part of an implant set according to a second embodiment of the invention;

FIG. 33 is a front perspective view of the complete implant set of the second embodiment of the invention;

FIG. 34 is a perspective view of the medial parts of the implant set of FIG. 32;

FIG. 35 is a top view of the medial parts of the implant set of FIG. 32;

FIG. 36 is a view from the medial side of the medial parts of the implant set of FIG. 32;

FIG. 37 is an anterior view of the medial parts of the implant set of FIG. 32;

FIG. 38 is a section on line A-A of FIG. 36;

FIG. 39 is a section on line B-B of FIG. 35;

FIG. 40 is a perspective view of the lateral parts of the implant set of FIG. 33;

FIG. 41 is a top view of the lateral parts of the implant set of FIG. 33;

FIG. 42 is a view from the lateral side of the lateral parts of the implant set of FIG. 33;

FIG. 43 is an anterior view of the lateral parts of the implant set of FIG. 33;

FIG. 44 is a posterior view of the lateral parts of the implant set of FIG. 33;

FIG. 45 is a section on line A-A of FIG. 42;

FIG. 46 is a section on line B-B of FIG. 41; and

FIG. 47 is a top view of the tibial components of the implant set of FIG. 42 when implanted showing articulation movement of the femur on the tibia.

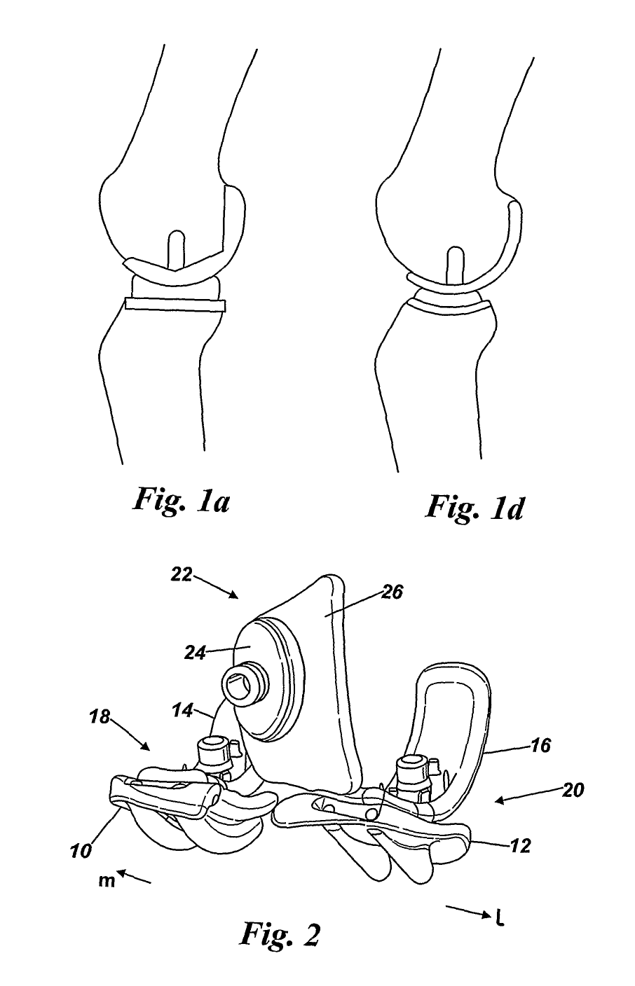

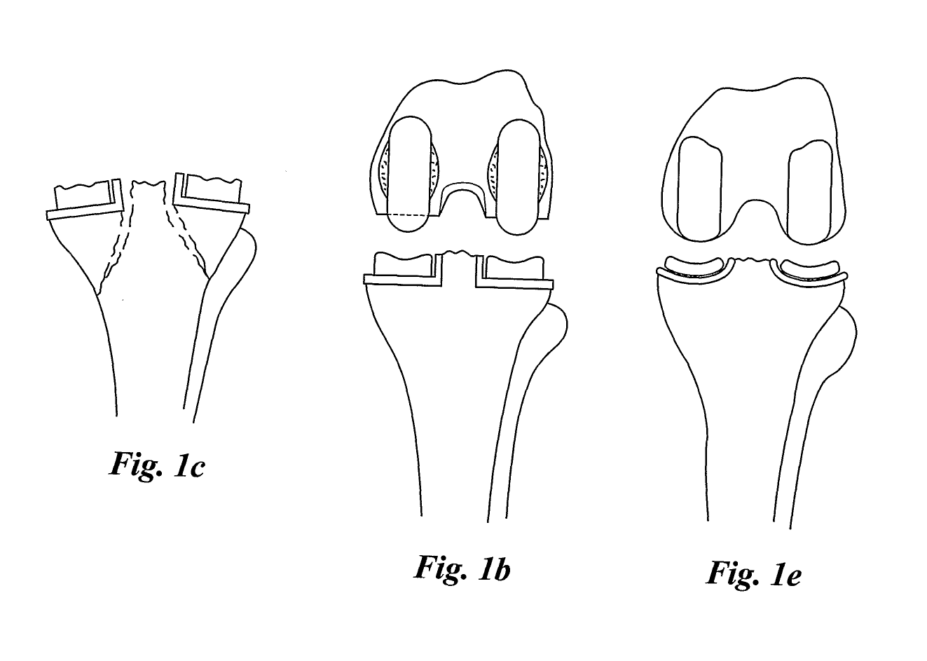

### DESCRIPTION OF THE PREFERRED EMBODIMENTS

Referring to FIGS. 2, 3 and 4, a modular knee implant set comprises medial and lateral tibial components 10, 12, medial and lateral femoral components 14, 16. The medial tibial and femoral components together form a medial bearing 18, and the lateral tibial and femoral components together form a lateral bearing 20. The implant set further comprises a patello-femoral bearing 22 comprising a patella component 24 and a trochlear component 26.

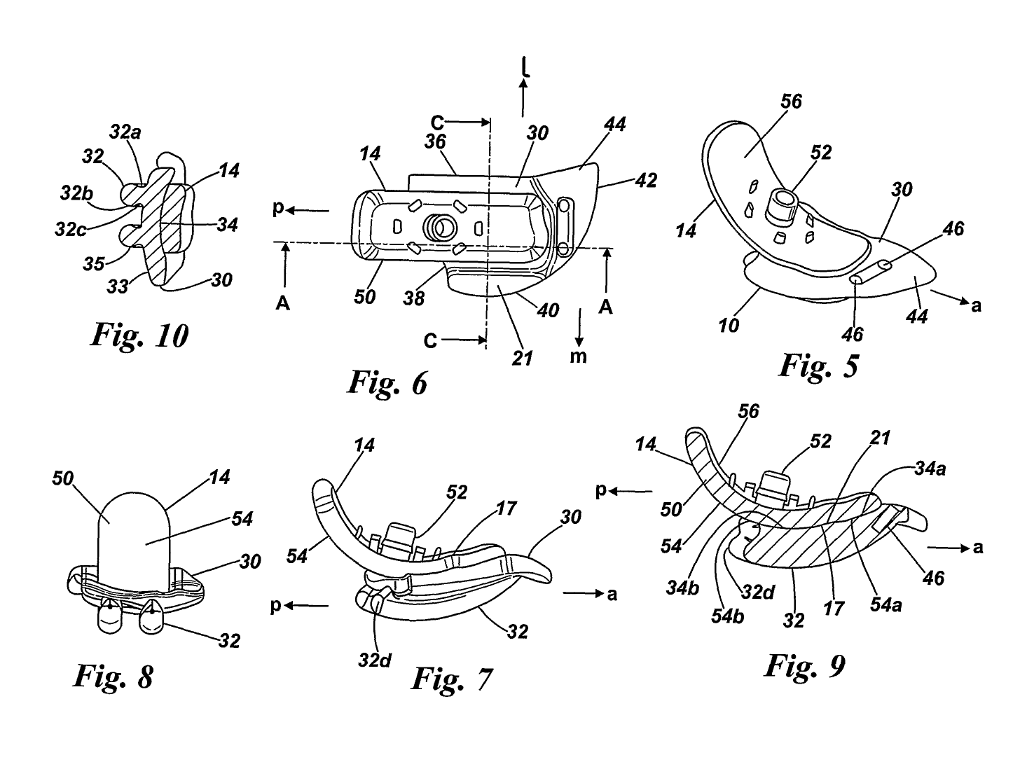

Referring to FIGS. 5 to 10, the medial bearing will now be described in more detail. The tibial component 10 comprises a main platform 30 with a pair of securing ribs or rails 32 on its underside 33 and a bearing surface 34 on its upper side. The bearing surface 34 is curved and the underside 33 of the platform 30 is similarly curved so that the platform is of generally uniform thickness.

As can best be seen in FIG. 6, the lateral edge 36 of the platform 30 is straight over most of its length. The posterior edge 38 is curved, with the lateral side of the platform extending further in the posterior direction than the medial side, and forms an abutment surface arranged to abut against the rear side of a recess formed in the tibia. The medial side 40 and front 42 of the platform 30 are formed as a continuous curve, and the front portion 44 of the platform forward of the bearing surface 34, is angled downwards to follow the front part of the top of the tibia. As can best be seen in FIGS. 5 and 9, a tool engagement formation in the form of a pair of parallel bores 46 is formed in the front portion 44 which are arranged to engage with an insertion tool used to insert the implant during surgery.

The bearing surface 34 of the medial tibial component has two bearing areas each of which has a constant radius of curvature in the sagittal plane, but with the two bearing areas having different radii of curvature. Specifically these areas comprise an anterior bearing area 34a and a posterior bearing area 34b, with the anterior bearing area 34a having the larger radius of curvature. These areas 34a, 34b are separated by a blending area 21 where the radius of curvature transitions smoothly from one area 34a to the other 34b. This blending area is narrow in the sagittal plane so as to maximize the lengths of the constant curvature areas 34a, 34b. For example it may be less than 10% of the length of the total bearing surface 34 in the sagittal plane. This blending zone 21 complements the blending zone of the femoral component (described below). When the knee is in full extension the load is spread across both the anterior and posterior bearing areas 34a, 34b and when flexed there is a large congruent contact area that is posterior to the transverse blending zone 21.

In each of the bearing areas 34a, 34b, there is a common centre of curvature for the bearing surface and the distal surface (underside) 33 below the bearing, thus giving a constant thickness bearing region of the component. The two bearing areas 34a, 34b could have a common centre of curvature, but preferable have different centres of curvature to allow a smooth transition between the two areas 34a, 34b. Anteriorly, the bone contact surface 33 is angled away from the bearing surface and acts to limit its posterior motion in the bone and thus enhancing fixation.

The securing ribs 32 are parallel to each other and extend in the anterior-posterior direction. The ribs 32 are curved with a constant radius of curvature along their length, being curved upwards towards their ends. They also have a narrow neck 32a supporting a wider locking portion 32b having a widest point (in the medial-lateral direction) 35 which is spaced vertically downwards from the underside 33 of the platform 30. The securing ribs 32 are therefore undercut on each side, with the upper part of the locking portion 32b forming a bearing surface 32c which forms an overhang and which is angled partially upwards towards the underside 33 of the platform 30. This forms a space between the locking portions 32b and the underside 33 of the platform into which a part of the bone can extend when the implant is inserted. This means that the securing ribs 32 can be slid into undercut grooves in the tibia to lock the implant in place as will be described in more detail below. Also the locking portion 32b of the ribs 32 extends posteriorly beyond the posterior end of the neck portion 32a, forming a posterior projection 32d, which is arranged to fit under a posterior undercut in the bone to provide further securing of the implant. As can best be seen in FIG. 7, the securing ribs 32 get shallower, projecting less far below the underside 33 of the bearing platform, towards their anterior end. This means that the bearing surfaces 32c get closer to the underside 33 of the bearing platform towards the anterior end of the implant. This means that, as the implant is inserted into the bone, the underside 33 of the bearing surface is pulled down onto the upper surface of the bone.

The medial femoral implant 14 comprises a main bearing portion 50 which is very generally of a rectangular shape being longer in the anterior-posterior direction than in the medial-lateral direction, and curved along its length so that its outer surface 54 forms a bearing surface arranged to slide over the bearing surface 34 of the medial tibial implant 10. A fixation post 52 projects upwards from the centre of the upwardly facing inner surface 56 of the femoral implant 14 which is arranged to secure the implant in place on the medial condyle of the femur. Optionally, other fixation designs may be used, including multiple posts, ribs or blades.

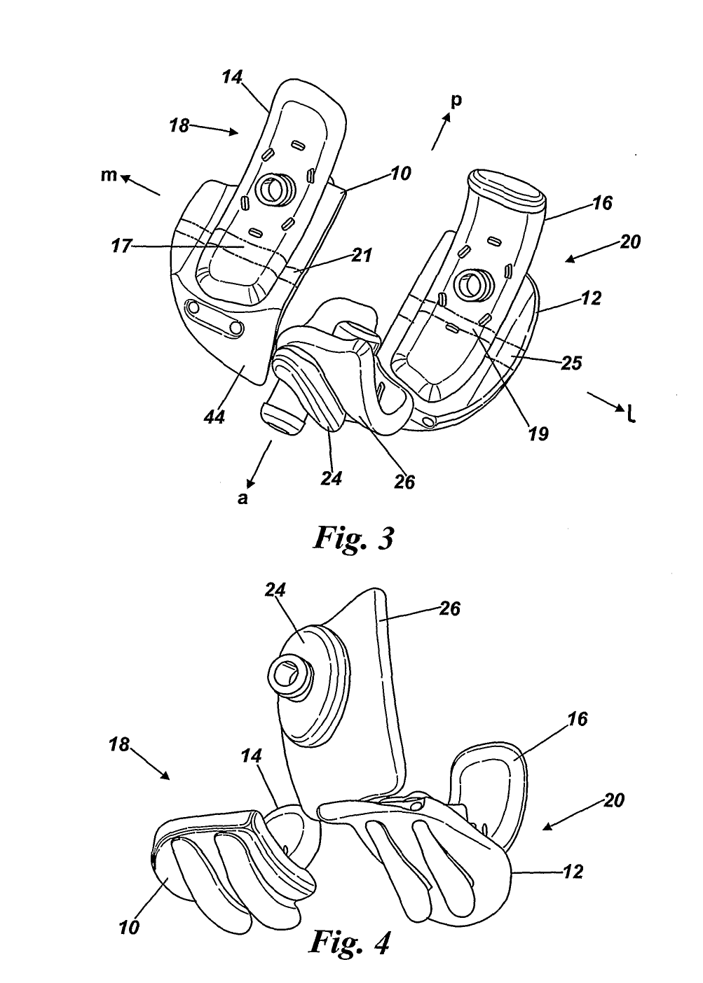

With reference to FIGS. 3, 7 and 9, a dual radius profile is present in the medial (and lateral) femoral component. The bearing surface 54 has an anterior bearing area 54a and a posterior bearing area 54b, with the anterior area 54a having a larger radius of curvature than the posterior area 54b. The break point or blending portion 17 between the two bearing areas marks the position or line at which two radii blend into each other. As with the tibial components, this blending portion 17 is narrow to maximize the constant radius bearing areas 54a, 54b, and in this case is less than 10% of the length of the bearing surface 54. The use of such a narrow blending zone provides a large bearing surface when the knee is in flexion and is heavily loaded and it also avoids the effects of having a large transition zone of intermediate radii that would otherwise preclude optimum contact.

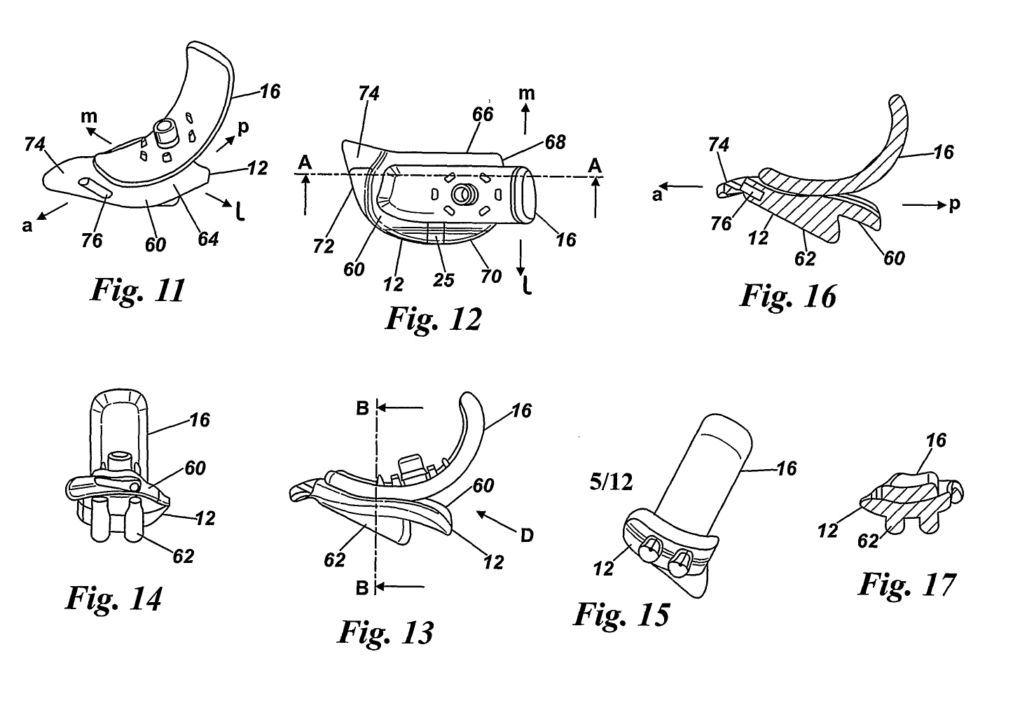

Referring to FIGS. 11 to 17, the lateral bearing will now be described in more detail. The tibial component 12 comprises a main platform 60 with a pair of securing ribs 62 on its underside and a bearing surface 64 on its upper side. The tibial articular surface 64 of the lateral tibial bearing is in two areas, an anterior area which is concave in the sagittal plane and then a posterior area that is convex in the sagittal plane. Both of these areas are concave in the coronal plane. The posterior area is thus an anticlastic or part-toroidal surface The concavity is congruent with the femoral component when the knee is extended and the convexity allows the femoral component to roll ‘down hill’ in a physiological fashion in flexion. In addition, the lateral tibial component has an anterior down-turned lip 74 for fixation.

The central undersurface of the tibial plateau components is curved in a medial lateral direction (i.e. in the coronal plane). This is in contrast to prior art systems where the bone is prepared by two perpendicular flat cuts. This avoids stress concentration and over cutting by saw blades. Both these are known causes of failure. This is most clearly seen in FIG. 17.

As can best be seen in FIG. 12, the medial edge 66 of the platform 60 is straight over most of its length The posterior edge 68, medial side 70 and front 72 of the platform 60 are formed as a continuous curve, and the front portion 74 of the platform is angled downwards to follow the front part of the top of the tibia. A pair of parallel bores 76 is formed in the front portion 74 which are arranged to engage with an insertion tool used to insert the implant during surgery.

The shape of the bone fixation fins on the under-surface uses the same principles as the medial bearing. The securing ribs 62 are again parallel to each other and extend in the anterior-posterior direction. The ribs 62 in this case are straight along their length. They have a similar cross section to the ribs 32 on the medial tibial implant, with a widest point (in the medial-lateral direction) 75 which is spaced vertically downwards from the underside 76 of the platform 60, and a partially upward facing surface, so that they can be slid into undercut grooves in the tibia.

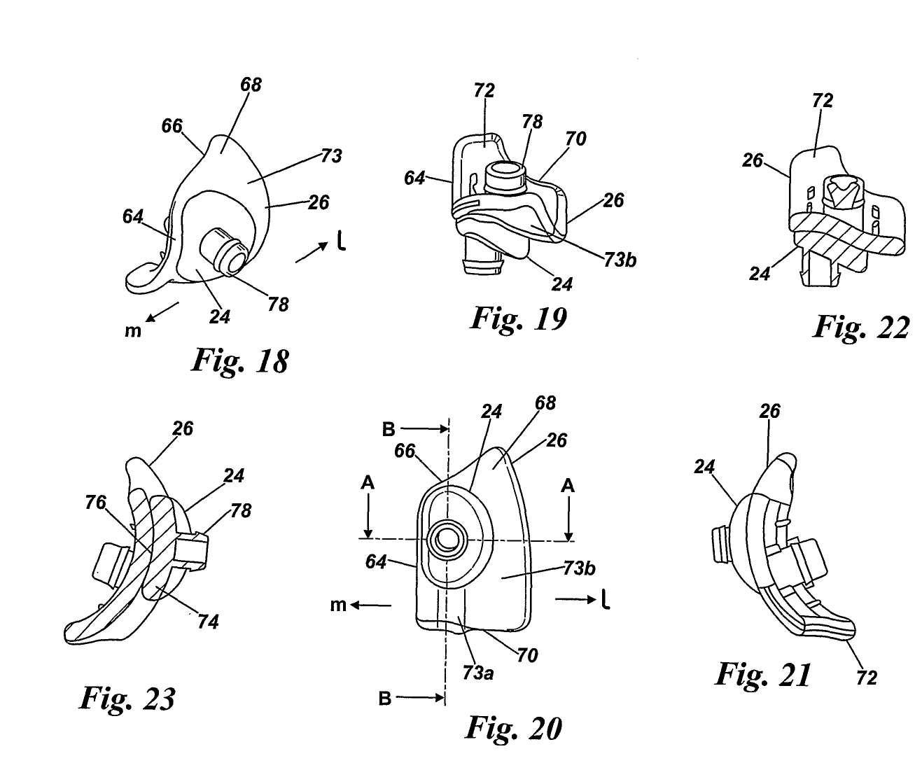

Referring to FIGS. 18 to 23, the patello-femoral bearing 22 comprises a patella component 24 and a trochlear component 26. The trochlear component 26 comprises a bearing platform of a generally constant thickness and curved so as to correspond to the front part of the femur over which the patella is located. A mounting post 60 or other fixation features projects from its concave rear surface 62 for mounting the component 26 on the femur. In shape the medial side 64 of the component is substantially straight and vertical, and the upper edge 66 is angled upwards towards the lateral side forming an upwardly projecting portion 68 on the lateral side. The lower edge 70 is angled upwards towards the lateral side, so that there is a downward projecting portion 72 on the medial side. The anterior bearing surface 73 of the trochlear component resembles only a part of the articular surface of the natural knee. The natural knee has two part-spherical articular surfaces, one medial, and one lateral, joined by a concave trochlear groove. However, the trochlear component bearing surface has a concave region 73a corresponding to the trochlear groove and a convex part spherical region 73b on the lateral side of the concave region 73a. On the medial side, the edge 64 of the bearing surface, and indeed of the component, is at a point where the bearing surface 73 is still concave. This means that there is no medial convex bearing surface on the medial side of the trochlear groove. This reflects the prevalent pattern of arthritic erosion affecting the lateral facet. The patella component 24 comprises a main bearing portion 74 with a bearing surface 76 which is convex on the medial side and concave on the lateral side, with no convex area on the medial side. A mounting post 78 or other fixation features are formed on the anterior side for mounting the component on the patella.

It is a feature of the design of the patello-femoral bearing that the components deliberately do not seek to replace the entire articular surface but are truncated to avoid the areas that are least affected by arthritic erosion, i.e. the medial part of the patello-femoral joint on both the femur and patella.

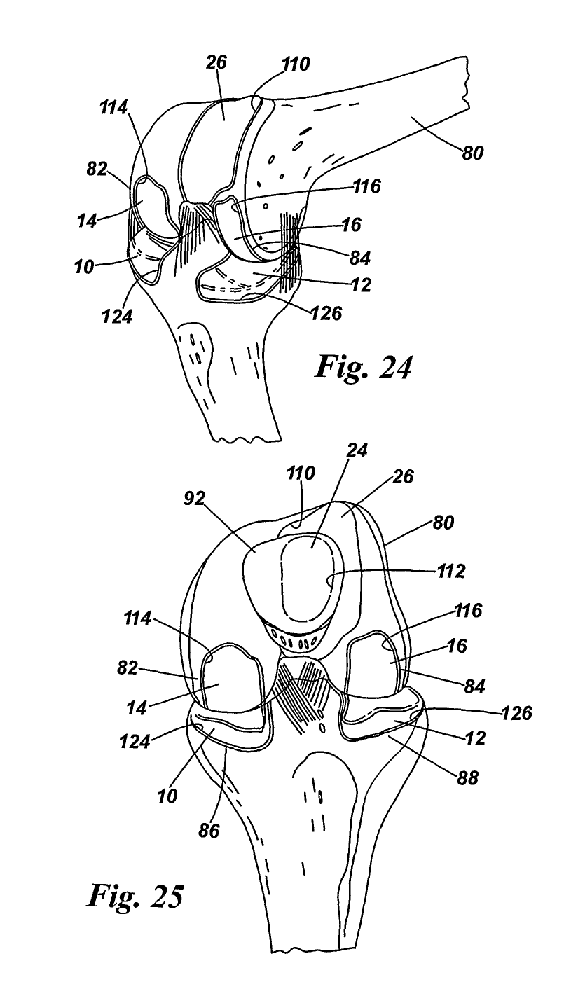

Referring to FIGS. 24 to 26, when the set of implants are in place in the knee joint, the femoral components 14, 16 of the medial and lateral bearings 18, 20 are located in the femur 80, in the medial and lateral femoral condyles 82, 84, and the tibial components 10, 12 are located in the medial and lateral tibial plateaux 86, 88. The trochlear component 26 of the patello-femoral bearing 22 is located in the anterior side of the trochlea 80 above the intercondylar notch 90, and the patella component 24 is mounted on the posterior-lateral side of the patella 92.

The implant set is arranged to cover the three areas mainly affected in primary osteoarthritis, and leave the original unaffected areas of bone in place. The main affected areas replaced are: the anteromedial aspect of the medial tibial plateau and its matching surface on the distal surface of the medial femoral condyle; the posterolateral aspect of the lateral tibial plateau and its matching surface on the posterior aspect of the lateral femoral condyle; and the lateral side of the patello-femoral joint, including the groove of the trochlea and the median ridge of the patella.

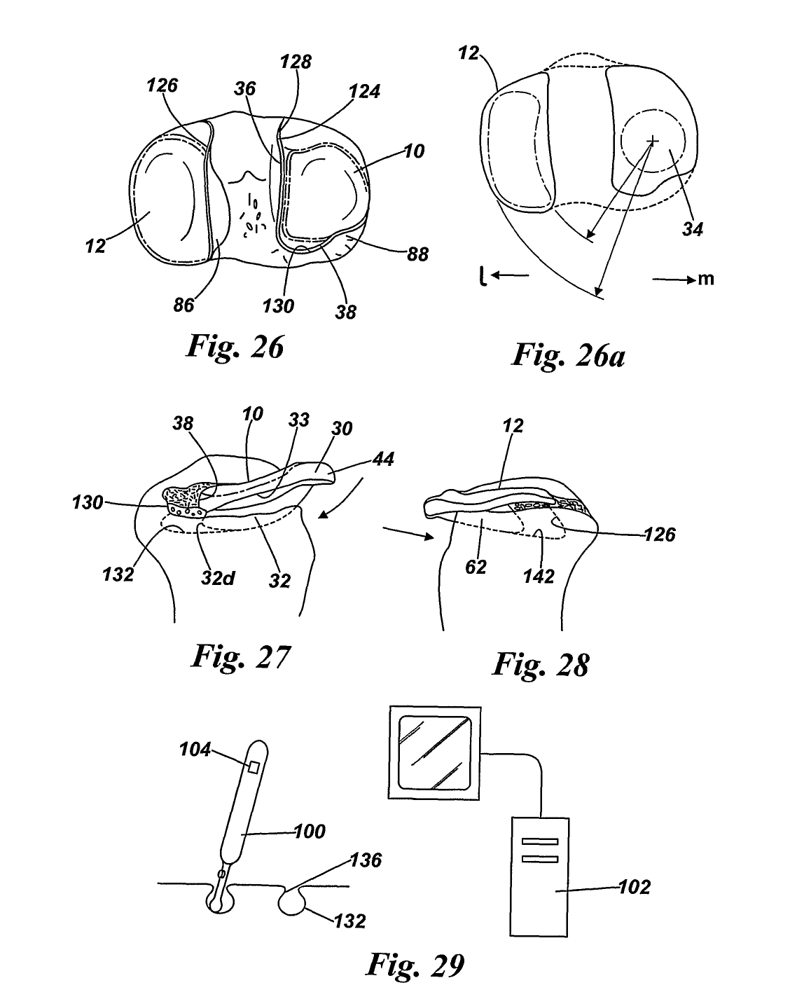

Referring to FIG. 26a, although the lateral tibial component 12 has a straight medial edge 66 the intention is that the femur should rotate in deep flexion with the axis of rotation at the centre of the medial bearing surface 34, hence the bearing surface 64 of the lateral component, which is concave in the coronal plane, is curved so as to provide a congruent bearing track that curves round towards the medial side at its anterior and posterior ends, with a centre of curvature at the centre of the medial bearing surface. The aim of the geometry is to ensure a congruent contact across the medial-lateral extent of the bearing surface whilst the femur is flexing and simultaneously externally rotating over the tibial surface and whilst the lateral condyle is descending the posterior slope.

The method of inserting the implants will now be described. Referring to FIG. 29, the bone is first sculpted using a burring tool 100 which is connected to a control system 102. The control system 102 uses position sensors 104 to monitor the position of the burring tool 100 and has a map stored in memory which defines parts of the bone which are to be cut away. The control system 102 then compares the position of the burring tool 100 with the map and controls it so that it will only cut away bone within the desired area. This allows the surgeon to control the burring tool 100 to perform the bone shaping, but limits his actions so that he will only cut the bone to the desired shape. A suitable system is the Acrobot Sculptor as discussed above.

The burring tool 100 is used to cut out individual recesses or pockets, one for each component of the implant set. Here it is assumed that the complete set is being used, although it will be appreciated that, for example, just one of the bearings comprising a pair of the components could be used. Referring to FIGS. 24 and 25, a trochlear implant pocket 110 is formed in the anterior femur. This is shaped to correspond to the shape of the patello-femoral implant component 26. The pocket 110 is offset to the lateral side of the femur 80. This will be oriented and positioned so that it resurfaces the lateral convex articular surface of the trochlea and the concave trochlear groove. It preserves the medial convex surface of the natural trochlea. A pocket 112 for the patella component 24 is formed in the posterior surface of the patella 92, again offset to the lateral side of the patella 92. This matches the most common pattern of painful arthritic erosions, which affect the lateral convex surface of the trochlea. Pockets 114, 116 are formed in the medial and lateral condyles 82, 84 to receive the condylar implants 14, 16. These pockets are of substantially constant depth over most of their area, with curved bases arranged to fit against the curved rear surfaces of the implants 14, 16. Fixing bores are also formed in the bottoms of these recesses to receive the fixing posts 52.

Pockets 124, 126 are formed in the medial and lateral tibial plateaux 86, 88 to receive the medial and lateral tibial components 10, 12. Referring to FIG. 26, the pocket 124 in the medial plateau has an approximately straight side 128 on the lateral side, and a curved side 130 at its posterior end, which is set in from the posterior edge of the medial tibial plateau 88, so that the lateral side and posterior end of the implant 10 can abut against these sides 128 130 when it is fully inserted. The medial and anterior sides of the pocket 124 are open as it extends to the medial and anterior sides of the medial tibial plateau 88. Referring to FIGS. 27 and 29two parallel retaining grooves 132 are cut into the bottom of the pocket 124, extending from the anterior end of the pocket in the posterior direction. These grooves 132 each have a narrow neck 136 near the surface and then open out below the surface, being undercut on each side, medial and lateral. They are also undercut at the posterior end to receive the posterior projection 32d on the ribs 32. The grooves 132 are curved along their length being higher at the ends than in the centre. The grooves 132 are shaped so as to receive the securing ribs 32 on the medial tibial component 10. As shown in FIG. 27 the medal tibial component 10 is inserted by placing the posterior ends of the ribs 32 in the anterior ends of the grooves 132, and then pushing the component along a curved path so that the ribs 32 slide along the grooves 132 until the implant is fully inserted. Insertion is performed using an insertion tool which engages with the formations 46. The inserter will engage in location features in the anterior surface of the tibial component (twin holes, slots etc) In the fully inserted position, the posterior edge 38 of the bearing platform 30 abuts against the posterior edge 130 of the pocket 124, the lateral edge 36 of the bearing platform 30 abuts against the lateral edge of the pocket 124, and the underside of the anterior portion 44 abuts against the tibia, along a surface formed with the burring tool 100 in the tibia. Also, since the ribs 32, and in particular their partially upward facing surfaces, converge with the underside 33 of the bearing platform towards the anterior end of the implant, the underside 33 is pulled down onto the bottom of the pocket 124 as the implant is inserted, so that in the fully inserted position the implant component 10 and the bone are in firm contact with each other.

Referring to FIGS. 26 and 28, the process of inserting the lateral tibial implant 12 is similar to that for the medial tibial implant 10. However in this case, while the medial side 140 of the pocket 126 is substantially straight so that the medial side of the implant 10 can abut against it, the pocket 126 extends all the way to the posterior side of the lateral tibial plateau 86, as well as its lateral and anterior sides. Also, so as to correspond to the shape of the securing ribs 62, the grooves 142 on the bottom of the pocket 126 are straight, extending from the anterior edge of the pocket 126 part way to the posterior edge of the lateral tibial plateau.

Again, the grooves 142 get deeper towards their posterior ends so that the implant 12 is pulled downwards onto the bottom of the pocket 126 as it is inserted.

It will be appreciated that, since distinct pockets or recesses are formed for each of the implant components in the femur, only the areas of bone which need to be replaced are replaced, and for example the rear edge 88 of the medial tibial plateau is left intact. Also each of those components can be replaced, with associated re-shaping of the pocket if required, without the need to replace the entire set of implants. Since the pockets have edges against which the implants fit, this provides good fit and fixation because sideways movement and rotation are prevented, eliminating the need for bone cement. Since the underside of the implants is pulled down hard onto the bone, the bone can easily grow to become attached to the implant to further secure it in place.

Referring back to FIGS. 26 and 26a, it is the rotational movement of the femur over the tibia, centred on a point in the medial tibial plateau, which allows the medial tibial component 10 to be shorter in the a-p direction than the lateral component 12. Since the lateral condyle of the femur moves in the a-p direction to some extent, the lateral tibial component 12 needs to cover the whole of the lateral tibial plateau, whereas the medial component 10 extends from the anterior edge of the medial tibial plateau, where it is inserted, only part of the way across the medial plateau towards the rear edge of the plateau. The posterior portion of the plateau can therefore be left in place as described above, thus reducing the amount of bone removed and helping to locate and secure the implant in place.

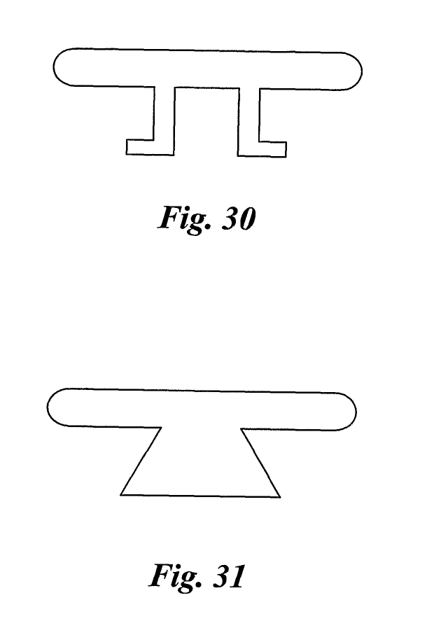

Referring to FIG. 30, in a further embodiment of the invention, the securing ribs 232 on the back of the tibial components are of an L-shaped cross section, having vertical portions 232a and horizontally projecting locking portions 232b. The locking surfaces 232c are formed on the upper side of the locking portions 232b.

Referring to FIG. 31, in a further embodiment, there is only one securing rib 332 on the back of the tibial components, which is of a dovetailed shape, having flat sides 332c which form the locking surfaces and face partially upwards towards the main platform 330.

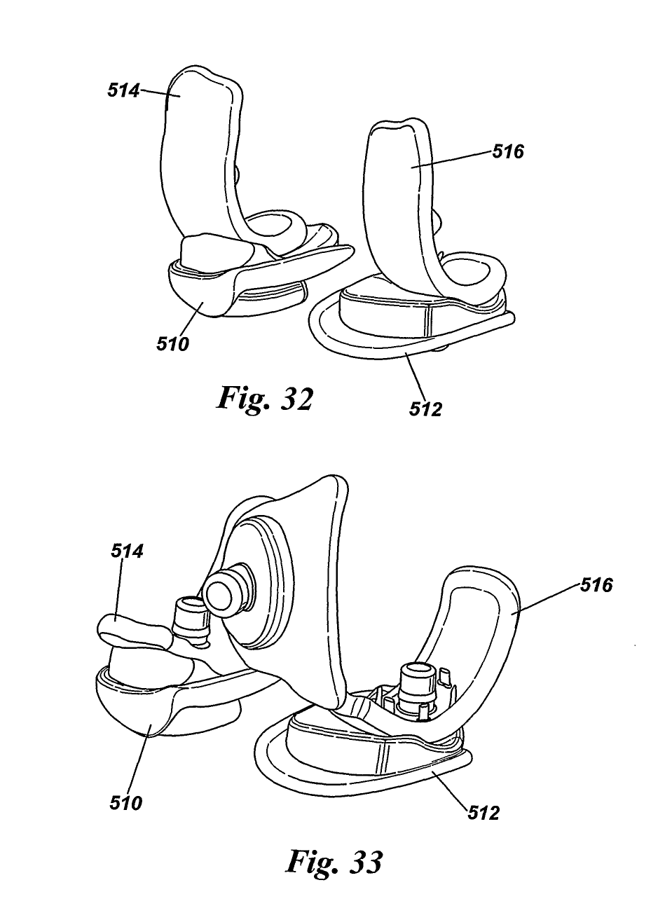

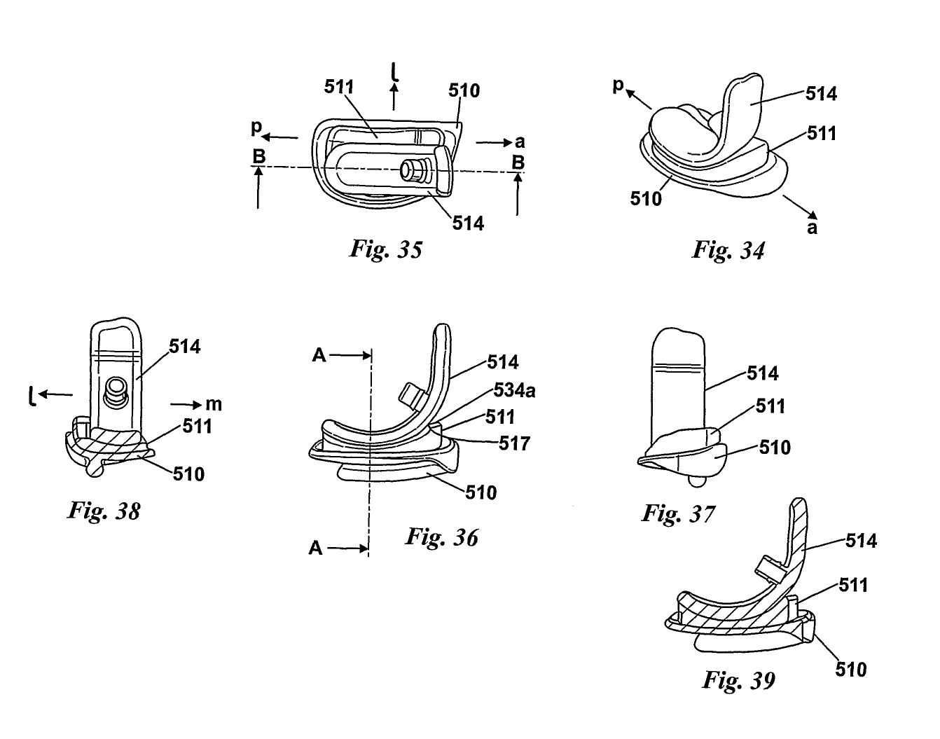

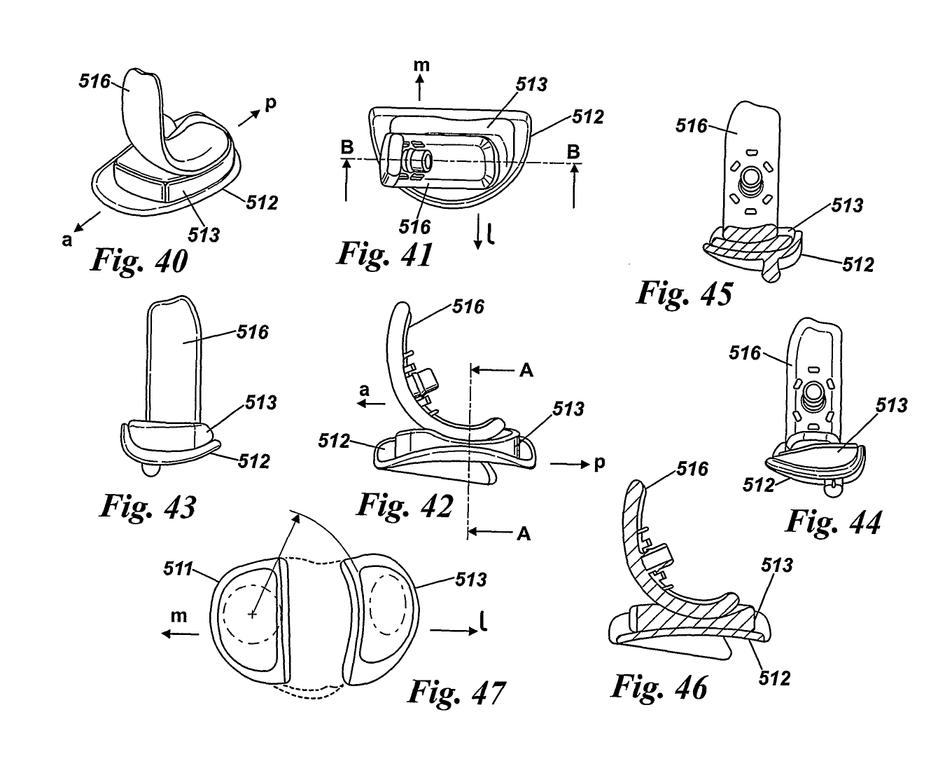

Referring to FIGS. 29 to 40 the principal features of the bearing surfaces for mobile (meniscal) bearing variant are in some ways similar to the previous fixed bearing embodiment, and corresponding features are indicated by the same reference numerals increased by 500. However, there are three components on each of the medial and lateral sides of the knee: femoral 514, 516, tibial 510, 512 and meniscal 511, 513, the latter being placed between the others. Both the upper and lower articulation surfaces of the meniscal bearings 511, 513 are fully congruent with the cooperating bearing surfaces on the mating metallic femoral and tibial component respectively. This is ensured by having, for each pair of contacting surfaces, a constant and equal radius of curvature in the sagittal plane for both mating surfaces and a constant and equal radius of curvature in the coronal plane for both mating surface. It will be appreciated that, for each pair of mating surfaces, the radii of curvature in the sagittal and coronal planes may be different from each other, and in fact this is an advantage as it inhibits rotation of the components relative to each other.

On the medial side, the bearing surface of the tibial component 510 is concave in the sagittal and coronal planes and so the underside of the meniscal bearing 511 has a matching convexity in both planes. The concavity in the sagittal plane will help to ensure stability of the knee.

The bearing surface on the femoral component 514 is convex in the saggital and coronal planes, and the top surface of the meniscal bearing is correspondingly concave in both planes.

On the lateral side, the upper bearing surface of the tibial component 512 is anticlastic, being convex in the sagittal plane but concave in the coronal plane, so the matching meniscal bearing 513 is also anticlastic and has an underside which is concave in the sagittal plane and convex in the coronal plane. This geometry in the sagittal plane promotes range of motion; by allowing the meniscal component 513 to ‘slide downhill’, it slackens the adjacent ligaments.

Both medial and lateral sides have a curved shape in the coronal plane, on the upper surface of the tibial component and the underside of the meniscal component, that remains constant from anterior to posterior. Thus the bearing remains congruent as the meniscal component slides backwards and forwards over the tibial component, when the knee flexes-extends.

The femoral components 514, 516 each have two bearing areas as in the first embodiment and the transition zone, between the bearing surface areas on the femoral components (both medial and lateral) is intended to come into contact with a transverse ridge 517 at the anterior edge of the concave upper bearing area 534 of the meniscal component when the knee reaches full extension. This feature helps to prevent knee hyperextension.

On the medial side, the femoral-meniscal bearing, i.e. the main upper bearing surface of the meniscal component 511, 513 will have a generally part-spherical geometry on the medial side, i.e. having equal radius of curvature in the sagittal and coronal planes, allowing the knee to rotate while maintaining congruent contact. On the lateral side, the geometry may also be part-spherical, but there can in some cases be an advantage to have a smaller radius in the coronal plane than in the sagittal plane, which will tend to ensure that the meniscal component remains aligned under the contact forces from the femoral component and does not tend to spin out of articulation.

FIG. 39 shows that the meniscal bearing surface of the medial tibial component is prolonged anteriorly, to increase the surface area of contact with the meniscal bearing. This means that the component gets thicker anteriorly, because the bearing surface extends over a major part of the downwardly angled undersurface that provides the location feature.

Referring to FIG. 47, as with the first, fixed bearing, embodiment, the implants are arranged to allow the femur to rotate about the centre of the bearing surface on the medial tibial component 510, and therefore, like the upper bearing surface of the lateral tibial component in the first embodiment, the anticlastic upper bearing surface on the lateral tibial component 512 is curved around to form a channel that is curved towards the medial side at its anterior and posterior ends. The under surface of the lateral meniscal component is similarly curved so that the miniscal component 513 can move in an arc to accommodate a slight rotation of the femur relative to the tibia during flexion.

1. An implant for bone re-surfacing in a joint, the implant comprising:
a bearing platform having a front surface which forms a bearing surface and a back surface; and
securing means projecting from the back surface, the securing means having a locking surface arranged to bear against an undercut surface of the bone to secure the implant against the bone.

2. An implant according to claim 1, wherein:
said locking surface is angled at least partly towards the back surface of the bearing platform so that a space is defined between the locking surface and the back surface into which a portion of the bone can extend to secure the implant against the bone.

3. An implant according to claim 1, wherein:
the locking surface extends in an insertion direction in which the implant can be moved to insert the implant.

4. An implant according to claim 1, wherein:
the securing means is in the form of a rib extending along the back of the bearing platform.

5. An implant according to claim 4, wherein:
one side of the rib is undercut so as to form an overhang so that a portion of the bone can project under the overhang so as to secure the implant to the bone.

6. An implant according to claim 1, wherein:
the securing means is straight so that the implant can be inserted in a straight line to secure it to the bone.

7. An implant according to claim 1, wherein:
the securing means is curved so that the implant can be inserted along a curved path to secure it to the bone.

8. An implant according to claim 1, wherein:
the securing means is one of a pair of securing means extending parallel to each other along the implant.

9. An implant according to claim 1, additionally comprising:
a tool engaging formation arranged to engage an insertion tool.

10. An implant according to claim 1, wherein:
the locking surface is angled relative to the back surface of the bearing platform so as to urge the back surface against the bone as the implant is inserted.

11. An implant according to claim 1, wherein:
said implant is a tibial implant arranged to resurface one of the medial and lateral tibial plateaux.

12. An implant according to claim 11, wherein:
the bearing platform has an anterior portion the upper surface of which is angled downwards relative to the bearing surface.

13. An implant according to claim 11, wherein:
the bearing platform has an anterior portion the back surface of which is angled downwards so as to abut against the bone when the implant is fully inserted.

14. An implant according to claim 11, wherein:
the bearing platform has an abutment edge at its posterior end arranged to abut against the edge of a recess in the tibia when the implant is fully inserted.

15. An implant according to claim 1, wherein:
at least a part of the back surface of the bearing platform is convex in a coronal plane.

16. An implant according to claim 1, wherein:
at least a part of the bearing surface is concave in a coronal plane.

17. An implant according to claim 1, wherein:
at least a part of the bearing surface is convex in a sagittal plane.

18. An implant according to claim 1, wherein:
at least a part of the bearing surface is concave in the sagittal plane.

19. An implant according to claim 1, wherein:
the bearing surface comprises an anterior area and a posterior area, the two areas having different radii of curvature.

20. An implant according to claim 19, wherein:
the radius of curvature of the anterior area is greater than the radius of curvature of the posterior area.

21. An implant according to claim 19, wherein:
the bearing surface includes a transition region between the anterior and posterior areas.

22. An implant according to claim 21, wherein:
the transition region takes up no more than 10% of the length of the bearing surface in the sagittal plane.

23. An implant according to claim 1, wherein:
the bearing surface and the back surface of the bearing platform are both curved so that the majority of the bearing platform is of a substantially constant thickness.

24. A uni-condylar implant set comprising:
a tibial implant comprising a bearing platform having a front surface which forms a bearing surface and a back surface, and securing means projecting from the back surface, the securing means having a locking surface arranged to bear against an undercut surface of the bone to secure the implant against the bone; and
a femoral implant.

25. An implant set according to claim 24, wherein:
the femoral implant comprises a femoral bearing portion having a bearing surface which is convex on a sagittal plane, the bearing surface having an anterior area and a posterior area, the areas having different radii of curvature.

26. An implant set according to claim 25, wherein:
the anterior area has a greater radius of curvature than the posterior area.

27. An implant set according to claim 25, wherein:
the bearing surface includes a transition region between the anterior and posterior areas.

28. An implant according to claim 27, wherein:
the transition region takes up no more than 10% of the length of the bearing surface in the sagittal plane.

29. An implant set according to claim 24, wherein:
the bearing surface of the femoral component is arranged to contact the bearing surface of the tibial component.

30. An implant set according to claim 24, further comprising:
a meniscal component arranged to be located between the tibial and femoral components and having upper and lower bearing surfaces arranged to contact the femoral and tibial components respectively.

31. An implant set according to claim 30, wherein:
the lower bearing surface of the meniscal component is convex in both a coronal plane and a sagittal plane.

32. An implant set according to claim 30, wherein:
the lower bearing surface of the meniscal component is convex in the coronal plane and concave in the sagittal plane.

33. An implant set according to claim 30, wherein:
the upper bearing surface of the meniscal component is concave in both a coronal plane and a sagittal plane.

34. A knee resurfacing implant set comprising:
two uni-condylar implant sets each of which is a uni-condylar implant set comprising a tibial implant comprising a bearing platform having a front surface which forms a bearing surface and a back surface, and securing means projecting from the back surface, the securing means having a locking surface arranged to bear against an undercut surface of the bone to secure the implant against the bone and a femoral implant.

35. An implant set according to claim 34, wherein:
one of the implant sets is a medial set and one of the implant sets is a lateral set, and the tibial component of the medial set is shorter in the anterior-posterior direction than the tibial component of the lateral set.

36. An implant set according to claim 35, wherein:
the tibial component of the medial set has an abutment surface at its posterior edge arranged to abut against a surface of a tibia to limit movement of the implant in the posterior direction.

37. A knee resurfacing implant set according to claim 34, further comprising:
a patella component for mounting on a patella and a trochlear component for mounting on the femur and having a bearing surface arranged to contact the patella component.

38. An implant set according to claim 37, wherein:
the bearing surface of the trochlear component comprises one convex part-spherical portion on its lateral side and a concave portion on its medial side.

39. An implant set according to claim 37, wherein:
the trochlear component comprises a bearing platform of substantially constant thickness, the front surface of which forms the bearing surface.

40. An implant set according to claim 39, wherein:
the bearing platform has at least one of an upward projection on its lateral side, and a downward projection on its medial side.

41. A method of resurfacing a bone comprising:
cutting an undercut groove in the bone;
providing an implant comprising a bearing portion with a back surface and securing means projecting from the back surface, the securing means having a locking surface arranged to bear against an undercut surface of the groove; and
inserting the securing means into the groove to secure the implant against the bone.

42. A method according to claim 41, wherein:
the implant is an implant comprising a bearing platform having a front surface which forms a bearing surface and a back surface; and securing means projecting from the back surface, the securing means having a locking surface arranged to bear against an undercut surface of the bone to secure the implant against the bone.

43. A method according to claim 41, wherein:
the securing means is straight and the implant is inserted in a straight line to secure it to the bone.

44. A method according to claim 41, wherein:
the securing means is curved and the implant is inserted along a curved path to secure it to the bone.

45. A method according to claim 41, wherein:
the implant is inserted into a tibial plateau from the anterior side in a posterior direction.

46. A method according to claim 41, further comprising:
cutting a pocket in the bone into which the implant is inserted.

47. A method according to claim 46, wherein:
the pocket has a side against which the implant abuts when it is inserted.

48. A unicondylar implant set comprising:
a tibial implant;
a femoral implant; and
a meniscal component arranged to be located between the tibial and femoral components and having upper and lower bearing surfaces arranged to contact the femoral and tibial components respectively, wherein the lower bearing surface of the meniscal component is convex in both a coronal plane and a sagittal plane.

49. A unicondylar implant set comprising:
a tibial implant;
a femoral implant; and
a meniscal component arranged to be located between the tibial and femoral components and having upper and lower bearing surfaces arranged to contact the femoral and tibial components respectively, wherein the lower bearing surface of the meniscal component is convex in the coronal plane and concave in the sagittal plane.

50. An implant set according to claim 48, wherein:
the upper bearing surface of the meniscal component is concave in both a coronal plane and a sagittal plane.

51. A knee resurfacing implant set comprising:
a patella component for mounting on a patella; and
a trochlear component for mounting on the femur and having a bearing surface arranged to contact the patella component wherein the bearing surface of the trochlear component comprises one convex part-spherical portion on its lateral side and a concave portion on its medial side.

52. An implant set according to claim 51, wherein:
the patella component has a bearing surface comprises one concave part-spherical portion on its lateral side and a convex portion on its medial side.

53. A method of partially resurfacing a bone comprising:
providing an implant;
cutting a recess in the bone having a shape corresponding to that of the implant; and
inserting the implant into the recess.

54. A method according to claim 53, further comprising:
providing a further implant, cutting a further separate recess in the bone having a shape corresponding to that of the further implant, and inserting the further implant into the further recess.

55. A method according to claim 54, wherein:
the recess is for a medial condylar implant and has a posterior end which is set in from the posterior edge of the medial tibial plateau.
